(19) United States
(12) Patent Application Publication
(10) Pub. No.: US 2003/0105442 A1
(43) Pub. Date: Jun. 5, 2003

(54) Convection of absorbent cores providing enhanced thermal transmittance

(76) Inventors: Lee W. Johnston, Suwanee, GA (US); John D. Litvay, Duluth, GA (US); Kenneth J. Molee, Suwanee, GA (US)

Correspondence Address: HUNTON & WILLIAMS, INTELLECTUAL PROPERTY DEPARTMENT, 1900 K STREET, N.W., SUITE 1200, WASHINGTON, DC, 20006-1109, US

(21) Appl. No.: 09/997,233

(22) Filed: Nov. 30, 2001

(51) Int. Cl. A61F 13/15; A61F 13/20

(52) U.S. Cl. 604/368; 604/378; 604/365

(57) ABSTRACT

The present invention generally relates to an absorbent article that includes a top sheet, a back sheet and an absorbent core disposed between the top sheet and the back sheet. The absorbent article has enhanced thermal transmittance by evincing a lower thermal resistance (clo) of less than about 1.7 watts/m², as measured in a Thermolabo apparatus. The absorbent article of the invention preferably has a low density, low basis weight core, and it provides improved comfort.

### FIELD OF THE INVENTION

The present invention relates generally to an absorbent core for a disposable absorbent garment, and more particularly to improved convection of an absorbent core comprising superabsorbent polymer (SAP) dispersed in a fibrous matrix. The absorbent garment of the invention provides improved comfort, and has enhanced thermal transmittance.

### BACKGROUND OF THE INVENTION

Traditionally, disposable absorbent garments such as infant diapers or training pants, adult incontinence products and other such products were constructed with a moisture-impervious outer backing sheet, a moisture-pervious body-contacting inner liner sheet, and a moisture-absorbent core sandwiched between the liner and backing sheets. Much effort has been expended to find cost-effective materials for absorbent cores that display favorable liquid absorbency and retention. Superabsorbent materials in the form of granules, beads, fibers, bits of film, globules, etc., have been favored for such purposes. Such superabsorbent materials generally are polymeric gelling materials that are capable of absorbing and retaining even under moderate pressure large quantities of liquid, such as water and body wastes, relative to their own weight.

The superabsorbent material generally is a water-insoluble but water-swellable polymeric substance capable of absorbing water in an amount at least ten times the weight of the substance in its dry form. In one type of superabsorbent material, the particles or fibers may be described chemically as having a back bone of natural or synthetic polymers with hydrophilic groups or polymers containing hydrophilic groups being chemically bonded to the back bone or in intimate admixture therewith. Included in this class of materials are such modified polymers as sodium neutralized cross-linked polyacrylates and polysaccharides including, for example, cellulose and starch and regenerated cellulose which are modified to be carboxylated, phosphonoalkylated, sulphoxylated or phosphorylated, causing the SAP to be highly hydrophilic. Such modified polymers may also be cross-linked to reduce their water-solubility.

It is known to provide absorbent laminates comprised of, for example, an upper layer, a lower layer, and a central fibrous layer containing from 30% to 95% by weight SAP. U.S. Pat. No. 6,068,620, the disclosure of which is incorporated herein by reference in its entirety and in a manner consistent with the present disclosure, discloses that the upper and lower layers are comprised of tissue, airlaid fluff pulp or synthetic non-woven fibrous layers. The upper and lower layers are said to provide thinner absorbent materials, and to assist in maintaining the integrity of the core, the laminate layered arrangement is said to minimize gel blocking, and the laminate can be folded in various configurations.

It generally is desirable to make absorbent articles as thin as possible, yet still thick enough to provide suitable absorption. H1565 proposes using a superabsorbent web made of superabsorbent particulate material in a continuous matrix of cellulose acetate fibers. The superabsorbent material is dispersed in the fibrous web by immobilizing it with a binder. Other documents disclosing incorporating superabsorbent polymers and various fibrous materials in absorbent cores include, inter alia, U.S. Pat. Nos. 5,350,370, and 5,436,066, the disclosures of each of which are incorporated by reference herein in their entirety.

Various materials used to make absorbent garments, as well as the thickness of its constituent parts can lead to high insulation, and consequently, less comfort. Various methods have been proposed to enhance an absorbent article's comfort. For example, many absorbent articles have extra elastic material to provide a better fit. H1969, H1,978 and H1,989 disclose a method of enhancing comfort by incorporating breathable microporous films having zoned breathability. The breathable films are said to have varying water vapor transmission rates (WVTR) in various zones to provide the requisite breathability. High WVTR levels in nonabsorborent areas are said to increase wearer comfort, which these documents allege can be measured by dry and wet heat transfer, and the permeability index.

Various mechanisms have been proposed in the past to determine the thermal resistance properties and moisture resistance properties of fabrics. For example, sweating hot plates have been used, which generally operate by maintaining a constant plate surface temperature, a known water vapor pressure at the surface of the plate, and constant environmental conditions, such as air temperature, humidity, and velocity. With the use of insulation and a surrounding guard, these traditional plates create a 1-dimensional flow of heat through test fabrics. Consequently, the thermal resistance and moisture resistance of fabrics can be calculated employing equations based on the recorded changing power input and changing water input used to maintain the plate surface at a selected temperature and water vapor pressure, respectively.

U.S. Pat. No. 5,749,259, the disclosure of which is incorporated by reference herein in its entirety, discloses a sweating hot plate and related method wherein the surface temperature of the plate is not constant but rather varies so that the plate, when used to test fabric placed thereon, will have a changing surface temperature that approximates the changing skin surface temperature of a human. Thus, the sweating hot plate disclosed in this document may be employed to predict the thermal comfort of the fabric directly.

### SUMMARY OF THE INVENTION

It would be desirable to provide an absorbent garment having an improved ability to retain fluids and consequently, to prevent leakage. It also would be desirable to provide an absorbent core that includes an increased amount of superabsorbent polymers to absorb greater amounts of fluids, but at the same time, is not appreciably thicker. It also would be desirable to provide an absorbent core that has the above mentioned characteristics, and in addition has improved thermal transmittance to improve the comfort of the garment.

It is therefore a feature of an embodiment of the invention to provide an absorbent garment having an improved ability to retain fluids. It is an additional feature of an embodiment of the invention to provide an absorbent garment that includes an absorbent core having SAP particles dispersed in a fibrous matrix, where the absorbent core has high dry and wet strength for processing and in-use performance. An additional feature of various embodiments of the invention is providing an absorbent core that has good fluid acquisition, distribution, and storage characteristics, preferably is thinner than conventional cores, and has improved thermal transmittance.

These and other features of the invention can be achieved by an absorbent article including a top sheet, a back sheet and an absorbent core disposed between the top sheet and the back sheet. The absorbent core of the invention is comprised of a fibrous layer containing superabsorbent polymer particles (SAP). The absorbent article has an intrinsic thermal resistance (Rcf) of less than about 0.20(Δ° C. m²/watt), as measured on a 20×20 inch sample in a Thermolabo apparatus. The absorbent article preferably has an Rcf of less than about 0.19 Δ° C. m²/watt, as measured on a 20×20 inch sample in a Thermolabo apparatus.

In accordance with an additional feature of an embodiment of the invention, there is provided an absorbent article including a top sheet, a back sheet and an absorbent core disposed between the top sheet and the back sheet. The absorbent core of the invention is comprised of a fibrous layer containing superabsorbent polymer particles (SAP). The absorbent article has a thermal resistance (clo) of less than about 1.7 w/m², as measured in a Thermolabo apparatus.

In accordance with an additional feature of an embodiment of the invention, there is provided a method of making an absorbent article that includes providing a topsheet material and a backsheet material. The method also includes preparing an absorbent core that includes SAP dispersed in a fibrous matrix, and disposing the absorbent core between the topsheet and the backsheet. The absorbent article made by the method has an intrinsic thermal resistance (Rcf) of less than about 0.25 Δ° C. m²/watt, as measured on a 20×20 inch sample in a Thermolabo apparatus, and an Rcf of less than about 0.19 Δ° C. m²/watt, as measured on a 20×20 inch sample in a Thermolabo apparatus. The absorbent article made by the method also preferably has a thermal resistance (clo) of less than about 1.7 w/m².

In addition to the foregoing advantages, the absorbent garment having the absorbent core with improved thermal transmittance improves the comfort of the garment by allowing body heat to more rapidly transmit to the outer environment. Further, due to the thinness of the resulting product, less packaging material is needed for the same amount of product, and shipping and handling costs are lowered.

These and other features and advantages of the preferred embodiments will become more readily apparent when the detailed description of the preferred embodiments is read in conjunction with the attached drawings.

### BRIEF DESCRIPTION OF THE DRAWINGS

FIG. 1 is a partially cut-away view of an embodiment of the present invention, shown with elastic members fully stretched in the main portion of the garment;

FIG. 2 is a cross-sectional view of the absorbent garment in FIG. 1 taken along line A-A, illustrating one embodiment for the absorbent laminate core of the invention;

FIG. 3 is a schematic illustration of an environmental chamber used in the Test Methods to determine the heat transfer characteristics of the absorbent article of the invention; and

FIG. 4 is a schematic illustration of various skin models used in the Test Methods to determine the heat transfer characteristics of the absorbent article of the invention.

### DETAILED DESCRIPTION OF THE PREFERRED EMBODIMENTS

As used herein, the terms “absorbent garment,” “absorbent article” or simply “article” or “garment” refer to devices that absorb and contain body fluids and other body exudates. More specifically, these terms refer to garments that are placed against or in proximity to the body of a wearer to absorb and contain the various exudates discharged from the body. A non-exhaustive list of examples of absorbent garments includes diapers, diaper covers, disposable diapers, training pants, feminine hygiene products such as feminine pads and adult incontinence products. Such garments may be intended to be discarded or partially discarded after a single use (“disposable” garments). Such garments may comprise essentially a single inseparable structure (“unitary” garments), or they may comprise replaceable inserts or other interchangeable parts.

The present invention may be used with all of the foregoing classes of absorbent garments, without limitation, whether disposable or otherwise. The embodiments described herein provide, as an exemplary structure, a diaper for an infant, however this is not intended to limit the claimed invention. The invention will be understood to encompass, without limitation, all classes and types of absorbent garments, including those described herein. Preferably, the absorbent core is thin in order to improve the comfort and appearance of a garment. The importance of thin, comfortable garments is disclosed, for example, in U.S. Pat. No. 5,098,423 to Pieniak et al., the disclosure of which is herein incorporated by reference in its entirety.

Throughout this description, the expressions “upper layer” and “lower layer” that refer to the layers surrounding the absorbent core of the invention are used merely to describe one layer above the core, and one layer below the core. The upper layer need not always remain vertically above the core, and the lower layer need not always remain vertically below the core. The upper and lower layer need not be included. Indeed, many embodiments of the invention encompass various configurations of the absorbent laminate core whereby the laminate is folded in such a manner that the upper layer ultimately becomes the vertically highest and vertically lowest layer at the same time. Other configurations are contemplated within the context of the present invention.

Throughout this description, the term “disposed” and the expressions “disposed on,” “disposing on,” “disposed in,” “disposed between” and variations thereof (e.g., a description of the article being “disposed” is interposed between the words “disposed” and “on”) are intended to mean that one element can be integral with another element, or that one element can be a separate structure bonded to or placed with or placed near another element. Thus, a component that is “disposed on” an element of the absorbent garment can be formed or applied directly or indirectly to a surface of the element, formed or applied between layers of a multiple layer element, formed or applied to a substrate that is placed with or near the element, formed or applied within a layer of the element or another substrate, or other variations or combinations thereof.

Throughout this description, the expression “tow fibers” relates in general to any continuous fiber. Tow fibers typically are used in the manufacture of staple fibers, and preferably are comprised of synthetic thermoplastic polymers. Usually, numerous filaments are produced by melt extrusion of the molten polymer through a multi-orifice spinneret during manufacture of staple fibers from synthetic thermoplastic polymers in order that reasonably high productivity may be achieved. The groups of filaments from a plurality of spinnerets typically are combined into a tow which is then subjected to a drawing operation to impart the desired physical properties to the filaments comprising the tow.

The present invention relates generally to absorbent articles, and in particular to an absorbent article that contains a topsheet, a backsheet, and an absorbent core disposed between the topsheet and the backsheet. The absorbent core of the invention is comprised of a fibrous layer containing superabsorbent polymer particles (SAP). The absorbent article has an intrinsic thermal resistance (Rcf) of less than about 0.25 Δ° C. m²/watt, as measured on a 20×20 inch sample in a Thermolabo apparatus, and an Rcf of less than about 0.19 Δ° C. m²/watt, as measured on a 20×20 inch sample in a Thermolabo apparatus. The absorbent article also preferably has a thermal resistance (clo) of less than about 1.7 w/m². It is preferred in the present invention that the Rcf of a 20 in.×20 in. sample of the absorbent article be less than about 0.185 Δ° C. m²/watt, more preferably less than about 0.182 Δ° C. m²/watt, and most preferably less than about 0.18 Δ° C. m²/watt. It also is preferred that the absorbent article have a thermal resistance (clo) of less than about 1.65 w/m², more preferably less than about 1.60 w/m² even more preferably less than about 1.53 w/m², and even more preferably less than about 1.47 w/m², and most preferably, less than about 1.40 w/m².

The absorbent article of the invention preferably has a front waist region, a rear waist region and a crotch region positioned between the front and rear waist regions. The front waist region and rear waist region can be associated with one another to form a waist opening, and two leg openings. Those skilled in the art recognize that “front” and “rear” in the context of the invention denote for clarity purposes only the front and rear of a user, and that the absorbent article could be reversed whereby the previously described “front” portion becomes the rear portion, and vice versa. For purposes of this description, however, the front generally denotes the front portion of the wearer, and it is understood that this is the portion of the garment that has the desirable thermal transmittance properties described herein.

Leg elastics preferably are provided along the leg openings for securely holding the leg openings against the thighs of the wearer to improve containment and fit. A fastening system, either resealable or permanent, preferably holds the absorbent article around the wearer's waist. The fastening system assists in associating the front waist region with the rear waist region. A pair of stand-up leg gathers or waist containment flaps may be attached to or formed from the body's side surface of the top sheet.

A particularly preferred embodiment of the absorbent article of the invention includes an absorbent laminate core comprising at least three layers. Each absorbent core laminate preferably comprises a high efficiency, SAP-containing central fibrous layer disposed between upper and lower tissue layers.

Other non-SAP-containing roll good materials such as latex or thermally bonded airlaid fluff pulp, (e.g., roll good available from Buckeye or Fort James), or synthetic spunbonded, carded, or hydro-entangled non-woven may be positioned above and below the absorbent core. In a particularly preferred embodiment of the invention, at least the central fibrous layer of the absorbent laminate core contains 30-95% by weight particulate or fibrous SAP and at least one other fibrous or particulate material that is capable of maintaining high SAP efficiency. As described in U.S. Pat. No. 6,068,620, SAP efficiency can be expressed as the ratio of the actual SAP absorbency under load, or AUL (expressed as grams of saline absorbed per gram of SAP in the laminate), and the maximum SAP AUL obtained under ideal conditions of low basis weight where gel blocking does not occur. SAP concentrations of 50-95% provide thinner roll good composites for efficient shaping and handling. High SAP concentrations also provide thinner absorbent cores that can provide new options for product design. The absorbent cores of the invention can be made using either a wet or dry process.

Forming the absorbent core of the invention with one or more inner layers disposed between an upper and lower layer is believed to decouple key performance attributes of traditional absorbent cores. As recognized by skilled artisans, the various layers of an absorbent core typically are designed with competing interests. A compromise usually is made at the sacrifice of the optimal performance attributes of each of the individual layers. By decoupling the performance attributes of the individual layers, the absorbent core of the preferred embodiments optimizes the key characteristic performance attributes of each of the laminated inner layers, thereby resulting in overall improved performance over previously known absorbent cores, or absorbent laminates.

Absorbent articles also typically are designed with competing thermal transmittance interests. Outer layers often are rendered breathable to allow more moisture and thermal energy to escape. The inner absorbent cores, however, are designed to retain moisture, which has a tendency to trap the thermal energy in the article. While not intending on being bound by any theory, the present inventors believe that the absorbent cores described herein, which preferably are relatively thin cores and comprised of low density materials and arranged in a configuration to have high SAP efficiency, provide improved thermal transmittance, when compared to conventional absorbent articles employing conventional absorbent cores. The improved thermal transmittance translates into an absorbent garment having improved comfort.

The invention now will be described with reference to the attached drawings illustrating preferred embodiments of the invention. For clarity, features that appear in more than one Figure have the same reference number in each Figure.

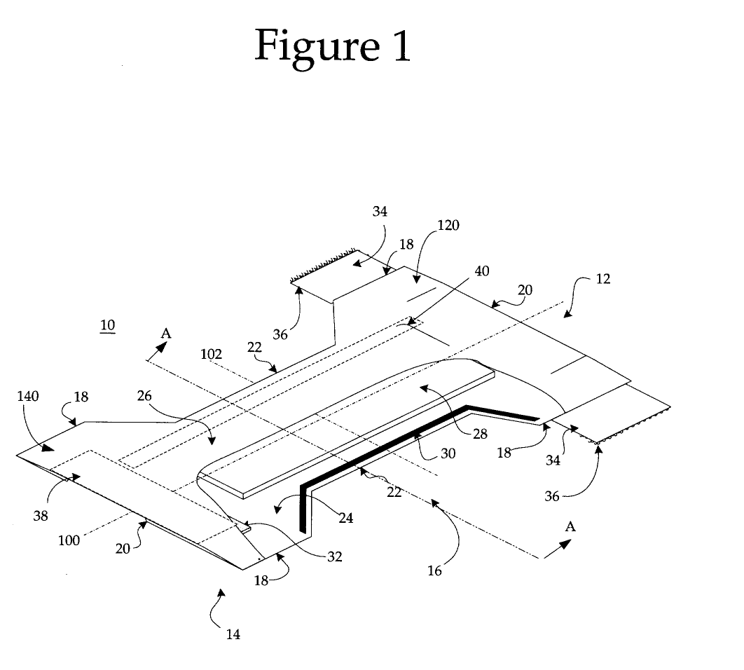

FIG. 1 is a partially cut away depiction of an exemplary embodiment of an absorbent garment 10 (preferably a disposable absorbent garment) of the present invention. The embodiment shown in FIG. 1 is an infant's diaper, however, this depiction is not intended to limit the invention, and those skilled in the art appreciate that the invention covers other types of absorbent articles. For simplicity, however, the invention will be described with reference to an infant's diaper. The garment 10 of FIG. 1 is depicted in a generally flattened position, with the body-facing side facing down, and with the various elastic components depicted in their relaxed condition with the effects of the elastics removed for clarity (when relaxed, the elastics typically cause the surrounding material to gather or “shirr”). In the flattened position, the garment 10 may have a generally hourglass shaped structure, but it may also have any other shape suitable for the given application, such as a rectangular shape, a trapezoidal shape, a “T” shape, and the like.

As used herein, the longitudinal axis 100 of the garment is the dimension of the garment corresponding to the front-to-rear dimension of the user, and the lateral axis 102 of the garment is the dimension corresponding to the side-to-side dimension of the user.

In use, the invention comprises a pant-like garment 10 having a waist-encircling region and a crotch region. The waist-encircling region may comprise a first waist region 12, disposed adjacent to, for example, the back waist region of a wearer's body, and a second waist region 14, disposed adjacent to, for example, the front waist region of a wearer's body. The first and second waist regions 12, 14, may correspond to the front and back of the wearer's body, respectively, depending on whether garment 10 is attached in front of or behind the subject wearer. The first and second waist regions are joined together at or near their lateral edges 18, causing the longitudinally distal edges 20 of the garment 10 to form the perimeter of a waist opening. A crotch region 16 extends between the first and second waist regions 12,14, and the crotch edges 22 form the perimeter of a pair of leg openings, when the garment 10 is placed on a subject wearer.

The garment 10 preferably comprises a topsheet 24, and a backsheet 26, which may be substantially coterminous with the topsheet 24. When the garment 10 is being worn, the topsheet 24 faces the wearer's body, and the backsheet 26 faces away from the wearer. An absorbent core 28 preferably is disposed between at least a portion of the topsheet 24 the backsheet 26. An embodiment of the present invention may further comprise various additional features. One or more pairs of elastic gathers 30 may extend adjacent the crotch edges 22. The garment 10 may also comprise one or more waste containment systems, such as inboard standing leg gathers 40, which preferably extend from the second waist region 14 to the first waist region 12 along opposite sides of longitudinal center line 100 (only one standing leg gather system 40 is shown in FIG. 1 for purposes of clarity). One or both of the first and second waist regions 12, 14 may also be equipped with strips of elastic waist foam 32 or other elastically extensible material, which help contract the garment around the wearer's waist, providing improved fit and leakage prevention.

The absorbent garment 10 also preferably includes fastening elements to enable attachment of the first waist region 12 to second waist region 14. Fastening elements preferably include a pair of tabs 34 that extend laterally away from opposite lateral edges 18 of the first waist region 12 of the garment 10. The tabs 34 may comprise an elastically extensible material (not shown), and may be designed to stretch around a wearer's waist to provide improved fit, comfort, and leakage protection. Such elasticized tabs 34 may be used in conjunction with, or in lieu of, waist foam 32, or other elastically extensible materials 32.

At least one fastening mechanism 36 (collectively referred to as “fastener 36”) is attached to each tab 34 for attaching the tab to the second waist region 14, thereby providing the garment 10 with a pant-like shape, and enabling garment 10 to be fixed or otherwise fitted on the wearer. The fasteners 36 may attach to one or more target devices 38 located in the second waist region 14.

Although not shown in the drawings, the absorbent garment 10 may also include grips attached along one of its edges proximal to each tab 34 to enable a caregiver to pull the grips, and not on the ends of the tabs 34, around the wearer and over the target devices 38 to thereby secure the fasteners 36 to the one or more target devices 38.

The various parts of the garment 10 can be attached to one another or associated with one another to form a structure that preferably maintains its shape during the useful life of the garment 10. As used herein, the terms “attached,” “joined,” “associated,” and similar terms encompass configurations whereby a first part is directly joined to a second part by affixing the first part directly to the second part, by indirectly joining the first part to the second part through intermediate members, and by fixing the relative positions of various parts by capturing parts between other parts. Those skilled in the art will appreciate that various methods or combinations of methods may be used to securely join the respective parts of the garment 10 to one another.

The topsheet 24 and backsheet 26 may be constructed from a wide variety of materials known in the art. The invention is not intended to be limited to any specific materials for these components. The topsheet 24 and backsheet can be shaped and sized according to the requirements of each of the various types of absorbent garment, or to accommodate various user sizes. In an embodiment of the invention in which the garment 10 is a diaper or an adult incontinence brief, the combination of topsheet 24 and backsheet 26, may have an hourglass shape, as seen in FIG. 1, or may have a rectangular, trapezoidal, “T” shape, or other shape.

Due to the wide variety of backing and liner sheet construction and materials currently available, the invention is not intended to be limited to any specific materials or constructions of these components. The back sheet 26 preferably is made from any suitable pliable liquid-impervious material known in the art. Typical back sheet materials include films of polyethylene, polypropylene, polyester, nylon, and polyvinyl chloride and blends of these materials. For example, the back sheet can be made of a polyethylene film having a thickness in the range of 0.02-0.04 mm. The backsheet 26 may be pigmented with, for example, titanium dioxide, to provide the garment 10 with a pleasing color or to render the backsheet 26 opaque enough that exudates being contained by the garment 10 are not visible from outside the garment. In addition, the backsheet 26 may be formed in such a manner that it is opaque, for example, by using various inert components in the polymeric film and then biaxially stretching the film. Other backsheet materials will be readily apparent to those skilled in the art. The backsheet 26 preferably has sufficient liquid imperviousness to prevent any leakage of fluids. The required level of liquid imperviousness may vary between different locations on the garment 10.

The backsheet 26 may further comprise separate regions having different properties. In a preferred embodiment, portions of the backsheet 26 are air-permeable to improve the breathability, and therefore comfort, of the garment 10. The different regions may be formed by making the backsheet 26 a composite of different sheet materials, chemical treatment, heat treatment, or other processes or methods known in the art. Some regions of the backsheet 26 may be fluid pervious. In one embodiment of the invention, the backsheet 26 is fluid impervious in the crotch 16, but is fluid pervious in portions of the first and second waist regions 12, 14. The backsheet 26 may also be made from a laminate of overlaid sheets of material.

The moisture-pervious top sheet 24 can be comprised of any suitable relatively liquid-pervious material known in the art that permits passage of liquid there through. Non-woven liner sheet materials are exemplary because such materials readily allow the passage of liquids to the underlying absorbent laminate core 28. Examples of suitable liner sheet materials include non-woven spunbond or carded webs of polypropylene, polyethylene, nylon, polyester and blends of these materials.

The backsheet 26 may be covered with a fibrous, nonwoven fabric such as is disclosed, for example, in U.S. Pat. No. 4,646,362 issued to Heran et al., the disclosure of which is hereby incorporated by reference in its entirety and in a manner consistent with this disclosure. Materials for such a fibrous outer liner include a spun-bonded nonwoven web of synthetic fibers such as polypropylene, polyethylene or polyester fibers; a nonwoven web of cellulosic fibers, textile fibers such as rayon fibers, cotton and the like, or a blend of cellulosic and textile fibers; a spun-bonded nonwoven web of synthetic fibers such as polypropylene; polyethylene or polyester fibers mixed with cellulosic, pulp fibers, or textile fibers; or melt blown thermoplastic fibers, such as macro fibers or micro fibers of polypropylene, polyethylene, polyester or other thermoplastic materials or mixtures of such thermoplastic macro fibers or micro fibers with cellulosic, pulp or textile fibers. Alternatively, the backsheet 26 may comprise three panels wherein a central poly backsheet panel is positioned closest to absorbent laminate core 28 while outboard non-woven breathable side backsheet panels are attached to the side edges of the central poly backsheet panel. Alternatively, the backsheet 26 may be formed from microporous poly coverstock for added breathability.

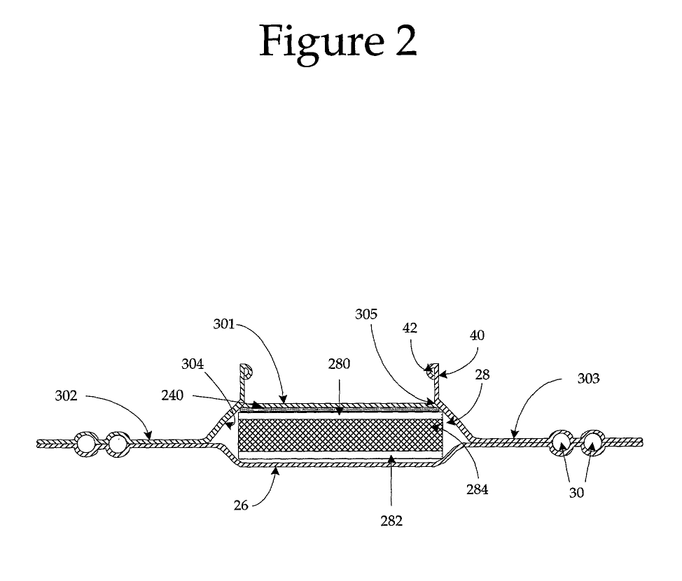

As illustrated in more detail in FIG. 2, the top sheet 24 may be formed of three separate portions or panels. Those skilled in the art will recognize, however, that top sheet 24 need not be made of three separate panels, and that it may be comprised of one unitary item. A first top sheet panel 301 may comprise a central top sheet panel formed from preferably a liquid-pervious material that is either hydrophobic or hydrophilic. The central top sheet panel 301 may be made from any number of materials, including synthetic fibers (e.g., polypropylene or polyester fibers), natural fibers (e.g., wood or cellulose), apertured plastic films, reticulated foams and porous foams to name a few. One preferred material for a central top sheet panel 301 is a cover stock of single ply non-woven material which may be made of carded fibers, either adhesively or thermally bonded, perforated plastic film, spunbonded fibers, or water entangled fibers, which generally weigh from 0.3-0.7 oz./sq. yd. and have appropriate and effective machine direction and cross-machine direction strength suitable for use as a baby diaper cover stock material. The central top sheet 301 panel preferably extends from substantially the second waist region 14 to the first waist region 12, or a portion thereof.

The second and third top sheet panels 302, 303 (e.g., outer top sheet panels), in this alternative embodiment may be positioned laterally outside of the central top sheet panel 301. The outer top sheet panels 302, 303 are preferably substantially liquid-impervious and hydrophobic, preferably at least in the crotch area. The outer edges of the outer top sheet panels may substantially follow the corresponding outer perimeter of the back sheet 26. The material for the outer top sheet portions or panels is preferably polypropylene and can be woven, non-woven, spunbonded, carded or the like, depending on the application.

The inner edges 304 (FIG. 2) of the outer topsheet portions or panels 302, 303 preferably are attached by, e.g., an adhesive, to the outer edges 305 of the inner topsheet portion or panel 301. At the point of connection with the outer edges 305 of the inner topsheet portion or panel 301, the inner edges 304 of the outer topsheet portions or panels 302, 303 extend upwardly to form waste containment flaps 40. The waste containment flaps 40 preferably are formed of the same material as the outer topsheet portions or panels 302, 303, as in the embodiment shown. They are preferably an extension of the outer topsheet portions or panels 302, 303.

The waste containment flaps 40 may be treated with a suitable surfactant to modify their hydrophobicity/hydrophilicity as desired, and they may be treated with skin wellness ingredients to reduce skin irritation. Alternatively, the waste containment flaps 40 may be formed as separate elements and then attached to the body side liner. In this alternative embodiment, the central topsheet portion or panel 301 may extend past the connection point with the waste containment flaps 40, and even extend to the periphery of the backsheet 26.

The waste containment flaps 40 preferably include a portion that folds over onto itself to form a small enclosure. At least one, and depending on the size of the enclosure sometimes more than one, elastic member 42 may be secured in the enclosure in a stretched condition. As has been known at least as long the disclosure of Tetsujiro, Japanese Patent document 40-11543, when the flap elastic 42 attempts to assume the relaxed, unstretched condition, the waste containment flaps 40 rise above the surface of the central topsheet portion or panel 301.

The topsheet 24 (as well as topsheet portions 301, 302, 303) may be made of any suitable relatively liquid-pervious material currently known in the art or later discovered that permits passage of a liquid there through. Examples of suitable topsheet materials include nonwoven spun-bonded or carded webs of polypropylene, polyethylene, nylon, polyester and blends of these materials, perforated, apertured, or reticulated films, and the like. Nonwoven materials are exemplary because such materials readily allow the passage of liquids to the underlying absorbent laminate core 28. The topsheet 24 preferably comprises a single-ply nonwoven material that may be made of carded fibers, either adhesively or thermally bonded, spunbonded fibers, or water entangled fibers, which generally weigh from 0.3-0.7 oz./sq. yd. and have appropriate and effective machine direction (longitudinal) and cross-machine (lateral) direction strength suitable for use as a topsheet material for the given application. The present invention is not intended to be limited to any particular material for the topsheet 24, and other topsheet materials will be readily apparent to those skilled in the art.

The topsheet 24 may further comprise several regions having different properties. In one embodiment of the present invention, the laterally distal portions of the topsheet 24, especially those used to make second and third top sheet panels 302, 303, preferably are substantially fluid impervious and hydrophobic, while the remainder of the topsheet 24 (e.g., central top sheet panel 301) is hydrophilic and fluid pervious. Different topsheet properties, such as fluid perviousness and hydrophobicity, may be imparted upon the topsheet 24 by treating the topsheet 24 with adhesives, surfactants, or other chemicals, using a composite of different materials, or by other means. The topsheet 24 may also be made from a laminate of overlaid sheets of material. The topsheet 24 also may be treated in specific areas like the crotch region, with skin wellness ingredients such as aloe, vitamin E, and the like.

As noted elsewhere herein, the topsheet 24 and backsheet 26 may be substantially coterminous, or they may have different shapes and sizes. The particular design of the topsheet 24 and backsheet 26 may be dictated by manufacturing considerations, cost considerations, and performance considerations. Preferably, the topsheet 24 is large enough to completely cover the absorbent laminate core 28, and the backsheet 26 is large enough to prevent leakage from the garment 10. The design of topsheet 24 and backsheet 26 is known in the art, and a skilled artisan will be able to produce an appropriate topsheet 24 and an appropriate backsheet 26 without undue experimentation.

The topsheet 24 and the backsheet 26 may be associated with one another using a variety of methods known in the art. For example, they may be thermally, ultrasonically, or chemically bonded to one another. They also may be joined using lines of hot melt adhesive or mechanical fasteners, such as thread, clips, or staples. In one embodiment, a hydrophilic adhesive, such as Cycloflex as sold by National Starch, a corporation headquartered in Bridgewater, N.J., is used to join the topsheet 24 to the backsheet 26. The particular joining method may be dictated by the types of materials selected for the topsheet 24 and backsheet 26.

As mentioned above, absorbent garment preferably is provided with leg elastics 30 extending through crotch region 16, adjacent crotch edge 22. The absorbent garment of the invention also preferably is provided with waist elastic material 32 optionally in the first and second waist regions, 12, 14, respectively, to enable and assist in stretching around the wearer. The waist elastics 32 may be similar structures or different to impart similar or different elastic characteristics to the first and second waist regions 12, 14 of the garment. In general, the waist elastics may preferably comprise foam strips positioned at the first and second waist regions 12, 14, respectively. Such foam strips preferably are about ½ to about 1½ inches wide and about 3-6 inches long. The foam strips preferably are positioned between the top sheet portions 24 or panels (301, 302, 303) and the back sheet 26. Alternatively, a plurality of elastic strands may be employed as waist elastics rather than foam strips. The foam strips preferably are comprised of polyurethane, but can be any other suitable material that decreases waist band roll over, reduces leakage over the waist ends of the absorbent garment, and generally improve comfort and fit. The first and optional second waist foam strips 32 preferably are stretched 50-150%, preferably 100% more than their unstretched dimension before being adhesively secured between the back sheet 26 and top sheet 24.

Each edge 22 that forms the leg openings preferably is provided with an adjacent leg elastic containment system 30. In the preferred embodiment, three strands of elastic threads (only two strands are shown in FIG. 2 for purposes of clarity) are positioned to extend adjacent to leg openings between the outer top sheet portions or panels 302, 303 and the back sheet 26. Any suitable elastomeric material exhibiting at least an elongation (defined herein as (L_{S}-L_{R})/L_{R} where L_{S} is the stretch length of an elastic element and L_{R} is retracted length, multiplied by 100 to obtain percent elongation) in the range of 5%-350%, preferably in the range of 200%-300%, can be employed for the leg elastics 30. The leg elastics 30 may be attached to the absorbent article 10 in any of several ways which are known in the art. For example, the leg elastics 30 may be ultrasonically bonded, heat/pressure sealed using a variety of bonding patterns, or glued to the garment 10. Various commercially available materials can be used for the leg elastics 30, such as natural rubber, butyl rubber or other synthetic rubber, urethane, elastomeric materials such as LYCRA (DuPont), GLOSPAN (Globe) or SYSTEM 7000 (Fulflex).

The fastening elements, preferably a fastening system 34 (e.g., tab 34) of the preferred embodiment, is attached to the first waist region 12, and it preferably comprises a tape tab or mechanical fasteners 36. However, any fastening mechanism known in the art will be acceptable. Moreover, the fastening system 34 may include a reinforcement patch below the front waist portion so that the diaper may be checked for soiling without compromising the ability to reuse the fastener. Alternatively, other absorbent article fastening systems are also possible, including tapes, adhesives, safety pins, buttons, and snaps.

As stated previously, the invention has been described in connection with a diaper. The invention, however, is not intended to be limited to application only in diapers. Specifically, the absorbent cores of the preferred embodiments may be readily adapted for use in other absorbent garments besides diapers, including, but not limited to, training pants, feminine hygiene products and adult incontinence products. Indeed, given the enhanced thermal transmittance, the absorbent articles of the present invention are particularly suitable for use in adult incontinence products and feminine hygiene products.

The underlying structure beneath the topsheet 24 may include, depending on the diaper construction, various combinations of elements, but in each embodiment, it is contemplated that the absorbent garment will preferably include an absorbent core 28. For example, an additional layer 280 may be disposed between the topsheet 24 and absorbent core 28, as shown in FIG. 2, and/or other additional layers may be disposed between these layers, or between absorbent core 28 and backsheet 26. The additional layer(s) 280 may include a fluid transfer layer, a fluid handling layer, a storage layer, a wicking layer, a fluid distribution layer, and any other layer(s) known to those having ordinary skill in the art.

Although the absorbent core 28 depicted in FIG. 2 has a substantially rectangular cross-sectional and plan view shape, other shapes may be used, such as a “T” shape or an hourglass shape. The shape of the absorbent core 28 may be selected to provide the greatest absorbency with a reduced amount of material. The absorbent core may be associated with the topsheet 24, backsheet 26, or any other suitable part of the garment 10 by any method known in the art, in order to fix the absorbent core 28 in place. In addition to the respective layers in the absorbent core 28, as will be described in greater detail hereinafter, the overall absorbent core 28 may be enclosed within a tissue wrapping, as disclosed in U.S. Pat. No. 6,068,620, the disclosure of which is incorporated by reference herein in its entirety. Skilled artisans are capable of designing and wrapping a suitable absorbent core 28 of the invention, using the guidelines provided herein.

The absorbent core 28 may extend into either or both of the first and second waist regions 12, 14. The absorbent core 28 of one preferred embodiment of the invention preferably includes at least three (3) layers whereby two of the layers are outer layers, (280, 282, FIG. 2), preferably outer tissue layers 280, 282, and one of the inner layers is a central fibrous layer 284 that preferably contains more than 50% by weight SAP.

Upper layer 280 and lower layer 282 can be made of any suitable material capable of containing the inner layer(s) of absorbent core 28. Preferably, upper layer 280 is hydrophilic and fluid pervious, and lower layer 282 is hydrophobic and fluid impervious. More preferably, upper layer 280 and lower layer 282 are comprised of the same tissue-like material.

In a preferred embodiment, the central fibrous layer 284 of absorbent core 28 comprises super absorbent polymer distributed within a fibrous structure. Central fibrous layers 284 of this type generally are known in the art, and exemplary absorbent cores are described in U.S. Pat. No. 6,068,620 and U.S. Pat. No. 5,281,207, both issued to Chmielewski, and U.S. Pat. No. 5,863,288, issued to Baker, the disclosures of each of which are herein incorporated by reference in their entirety and in a manner consistent with this disclosure.

Certain fibrous and particulate additives preferably are used as constituent elements of an absorbent core to maintain high SAP efficiencies when the SAP concentration is in the range of about 50-95%, more preferably about 60-90%, and most preferably about 75-85%. Super absorbent polymers of the surface cross-linked variety perform best in these laminates. The above-described particulate additives preferably are constituent elements of the central fibrous layer 284, and preferably include, but are not limited to, cellulose acetate fibers, rayon fibers, Courtauld's LYOCELL fibers, polyacrylonitrile fibers, surface-modified (hydrophilic) polyester fibers, surface-modified polyolefin/polyester bicomponent fibers, surface-modified polyester/polyester bicomponent fibers, cotton fibers, or blends thereof. Of the foregoing, cellulose acetate is the most preferred fibrous additive for use in central fibrous layer 284.

In addition, rayon, Courtauld's LYOCELL, polyacrylonitrile, cotton fibers and cotton linters have similar properties to cellulose acetate and are alternatively preferred. The remaining fibers, surface-modified polyolefin/polyester bicomponent fibers, and surface-modified polyester/polyester bicomponent fibers are also believed to be effective fibrous additives. To maintain high SAP concentrations, the concentration of fibrous additives in the central fibrous layer 284 of the absorbent core 28 of the invention preferably is about 5-50%, more preferably about 10-30%, and most preferably about 15-25%. Most preferably, the central fibrous layer 284 comprises from about 75-85% SAP and from about 15-25% fibrous additives selected from the foregoing group.

The fibrous component of the central fibrous layer 284 most preferably is a crimped tow of cellulose acetate or polyester. Alternatively, the fibrous component of the central fibrous layer 284 may be a low-density roll good made in a separate process. Still further yet, the fibrous component could also be a carded web formed on-line. Optionally, it is advantageous to introduce from about 1-5% of a thermally bondable fiber into the fibrous component of the central fibrous layer 284 for wet strength and core stability in use.

Particulate additives may be added to central fibrous layer 284 in addition to or as a substitute for the foregoing fibrous additives in order to maintain high SAP efficiency. The particulate additives preferably are insoluble, hydrophilic polymers with particle diameters of 100 μm or less. The particulate additives are chosen to impart optimal separation of the SAP particles. Examples of preferred particulate additive materials include, but are not limited to, potato, corn, wheat, and rice starches. Partially cooked or chemically modified (i.e., modifying hydrophobicity. hydrophilicity, softness, and hardness) starches can also be effective. Most preferably, the particulate additives comprise partially cooked corn or wheat starch because in this state, the corn or wheat are rendered larger than uncooked starch and even in the cooked state remain harder than even swollen SAP. In any event, regardless of the particulate additive chosen, one of the many important criteria is to use particulate additives that are hard hydrophilic materials relative to swollen SAP or which are organic or inorganic polymeric materials about 100 microns in diameter. Fibrous and particulate additives can be used together in these absorbent laminates. Examples of SAP/particulate and SAP/fiber/particulate additives include those described in, for example, U.S. Patent No. 6,068,620.

Any superabsorbent polymer (SAP) now known or later discovered may be used in central fibrous layer 284, so long as it is capable of absorbing liquids. Useful SAP materials are those that generally are water-insoluble but water-swellable polymeric substance capable of absorbing water in an amount that is at least ten times the weight of the substance in its dry form. In one type of SAP, the particles or fibers may be described chemically as having a back bone of natural or synthetic polymers with hydrophilic groups or polymers containing hydrophilic groups being chemically bonded to the back bone or in intimate admixture therewith. Included in this class of materials are such modified polymers as sodium neutralized cross-linked polyacrylates and polysaccharides including, for example, cellulose and starch and regenerated cellulose which are modified to be carboxylated, phosphonoalkylated, sulphoxylated or phosphorylated, causing the SAP to be highly hydrophilic. Such modified polymers may also be cross-linked to reduce their water-solubility.

Commercially available SAPs include a starch modified superabsorbent polymer available under the tradename SANWET® from Hoechst Celanese Corporation, Portsmouth, Va. SANWET® is a starch grafted polyacrylate sodium salt. Other commercially available SAPs include a superabsorbent derived from polypropenoic acid, available under the tradename DRYTECH® 520 SUPERABSORBENT POLYMER from The Dow Chemical Company, Midland Mich.; AQUA KEEP manufactured by Seitetsu Kagaku Co., Ltd.; ARASORB manufactured by Arakawa Chemical (U.S.A.) Inc.; ARIDALL 1125 manufactured by Chemdall Corporation; and FAVOR manufactured by Stockhausen Inc.; HYSORB manufactured by BASF Aktiengesellschaft, Ludwigshafen, Germany. A particularly preferred SAP is one designated P-7700, which is available from BASF, Ludwigshafen, Germany. Any of these SAP could be used in the invention either alone or in combination with one another, so long as the absorbent article has the thermal transmittance properties described herein.

In accordance with the present invention, improved absorbent articles are advantageously based upon continuous crimped filament tow, and accordingly, the central fibrous layer 284 is advantageously prepared there from. This fiber structure has high structural integrity, and as such, is distinct from a matrix of discontinuous fibers described as fluff in the prior art. The high structural integrity enables the production of stronger webs than those formed from discontinuous fibers, which in turn are believed to enable the production of thinner absorbent pads. In addition, the use of such fibers enables the production of ultra low density absorbent cores, when compared to absorbent cores prepared by dispersing SAP particles in fluff.

Beneficially, cellulose ester tow is used. Non-limiting examples of suitable cellulose esters include cellulose acetate, cellulose propionate, cellulose butyrate, cellulose caproate, cellulose caprylate, cellulose stearate, highly acetylated derivatives thereof such as cellulose diacetate, cellulose triacetate and cellulose tricaproate, and mixtures thereof such as cellulose acetate butyrate. A suitable cellulose ester will include the ability to absorb moisture, preferably is biodegradable, and is influenced not only by the substituent groups but also by the degree of substitution. The relationship between substituent groups, degree of substitution and biodegradability is discussed in W. G. Glasser et al, BIOTECHNOLOGY PROGRESS, vol. 10, pp. 214-219 (1994), the disclosure of which is incorporated herein by reference in its entirety.

Continuous filament tow useful in the present invention is beneficially moisture-absorbent and biodegradable. Accordingly, cellulose acetate tow is typically preferred for use in the invention. Typically, the denier per fiber (dpf) of the tow fiber will be in the range of about 1 to 9, preferably about 3 to 6. For the same weight product, filaments of lower dpf may provide increased surface area and increased moisture absorption. Total denier may vary within the range of about 20,000 to 60,000, depending upon the process used.

It is particularly preferred in the invention to use tow having crimped filaments. Crimp aids in opening. Separation of filaments resulting from bloom advantageously results in increased available filament surface area for superabsorbent material immobilization and increased moisture absorption. Gel blocking also may be reduced by using crimped tow in the fibrous absorbent core 284. As therefore may be understood, more crimp is typically better, with in excess of about 20 crimps per inch being usually preferred. Continuous filament, cellulose ester tow having crimped filaments with about 25 to 40 crimps per inch, is commercially available from Hoechst Celanese Corporation, Charlotte, N.C.

It is especially preferred in the present invention to prepare a relatively low density, low basis weight core material that is thin, when compared to conventional cores made with fluff pulp and SAP. Continuous cellulose acetate tow that has been opened using conventional opening procedures preferably is used as the fibrous component, and it is preferred to use the tow in an amount ranging from about 1% to about 45% by weight, preferably, from about 3% to about 30% by weight, and most preferably from about 12% to about 20% by weight, based on the combined weight of the fibrous material and SAP in the absorbent core. Most preferably, the cellulose acetate is used in an amount of about 17% by weight.

It is especially preferred to make the absorbent core material by first applying adhesive to one surface of a tissue layer having a basis weight within the range of from about 10 to about 30 grams/m², (“gsm”) most preferably, about 16 gsm. The opened continuous filaments of cellulose acetate tow, commercially available from Celanese Acetate, Charlotte, N.C., then preferably is supplied and deposited on the adhesive side of the tissue layer. Super absorbent polymer (SAP) particles, most preferably P-7700, available from BASF AG, Ludwigshafen, Germany, is disposed on and in the opened continuous filaments of cellulose acetate tow. The SAP is distributed in an amount ranging from about 55% to about 99% by weight, based on the combined weight of the fibrous material and SAP in the absorbent core, more preferably from about 70% to about 97% by weight, even more preferably from about 80% to about 88%, and most preferably about 83% by weight.

The absorbent core preferably is formed by disposing a second tissue layer on the central fibrous layer containing the mixture of SAP and tow fibers, which is deposited on a tissue layer. It is preferred that the second tissue layer have a basis weight within the range of from about 10 to about 40 grams/m², (“gsm”), more preferably from about 12 to about 30 gsm, and most preferably, about 16 gsm. It also is preferred that the first tissue layer be treated to render it hydrophobic, which may occur by virtue of application of adhesive, and the second tissue layer be hydrophillic, either naturally, or by treating with the appropriate surfactant, as is known in the art.

If desired, a superabsorbent, absorptive pad of multiple layer thickness, may be provided. To this end, the tow may be, for example, lapped or crosslapped in accordance with conventional procedures. In this way, a +l superabsorbent, absorptive material of a desired weight and/or thickness may be provided. The specific weight or thickness will depend upon factors including the particular end use.

The SAP may be provided in any particle size, and suitable particle sizes vary greatly depending on the ultimate properties desired. Those skilled in the art are capable of selecting a suitable particle size for use in the present invention, using the guidelines provided herein.

It has been known to prepare absorbent cores comprised of cellulose acetate tow or other polymeric fibers and SAP, as described in H1565, and U.S. Pat. Nos. 5,436,066, and 5,350,370, the disclosures of each of which are incorporated by reference herein in its entirety. It was conventional to add tackifying agents, specific size fibers, or specific fibers in combination with fluff, in order to prepare the absorbent core and immobilize the SAP particles. The present inventor believes that these additional materials may add to density of the core, or otherwise adversely affect the overall thermal transmittance of the absorbent article made there from. Thus, it is preferred not to use ethylene glycol, tackifying agents, and very small particulate fibers in the invention, although they may be used to the extent they do not increase the overall clo value of the absorbent article to about 2.0.

The total basis weight of the absorbent core 28 including fibrous materials, SAP, tissue, additional layers, and additives, can be anywhere from about 350-2000 grams per square meter (gsm), preferably from about 600 to about 1500 gsm, more preferably from about 650 to about 1350 gsm, and most preferably from about 750 to about 1000 gsm, the upper and lower limits of each and any of these ranges may be interchanged. The foregoing fibrous additives maintain high SAP efficiency at high SAP concentrations even when they are mixed with soft or hard wood fluff pulp fibers.

The density of the absorbent core 28 including fibrous materials, SAP, tissue, additional layers, and additives, preferably ranges from about 0.05 to about 0.45 g/cm³, more preferably from about 0.05 to about 0.3 g/cm³, even more preferably from about 0.06 to about 0.25 g/cm³, even more preferably from about 0.075 to about 0.11 g/cm³, and most preferably from about 0.08 to about 0.10 g/cm³. The density of the absorbent core material can be measured using standard techniques well known in the art.

Optionally, about 1-10%, preferably about 5%, by weight of thermally bondable synthetic fibers can be added to the absorbent core 28 to impart additional wet strength to the laminate. This will improve the stability of the core during use of the diaper. The preferred synthetic fibers are polyolefin/polyester fibers and polyester/polyester bicomponent fibers.

While, as discussed above, the present invention is premised in part on the discovery that certain fibrous and particulate additives maintain high SAP efficiencies when the SAP concentration is in the range of about 50-95%, fluff/SAP central fibrous layers 284 that contain greater than about 50% SAP may require additional structural or design measures to contain the SAP in the layer and provide adequate wet strength for overall core stability in manufacture and use. One solution is to adhesively or thermally bond the absorbent material to improve wet strength and core stability. This, unfortunately, results in slower than adequate rates of absorption and poor SAP efficiency. Another solution resides in the discovery that a high SAP concentration central fibrous layer 284 may be hydrogen bonded to additional fibrous layers. When a highly concentrated SAP-containing central fibrous layer 284 is hydrogen bonded to upper and lower layers 280, 282, or optionally is wrapped by a fibrous layer (not shown), the SAP efficiency is not impaired, wet strength increases, and the upper and lower layers 280, 282, and optional wrapping layer add stability to the core during manufacture. The structure and composition of the absorbent cores 28 preferably are designed for optimal strength, SAP containment, and liquid distribution. Skilled artisans are capable of designing absorbent cores 28 to optimize the foregoing attributes, using the guidelines provided herein.

Depending on whether a wet or dry process is used to make the absorbent cores 28, bonding central fibrous layer 284 with tissue layers 280, 282, can be achieved with hydrogen or adhesive bonds. If the material used to form the absorbent cores 28 contains about 1-5% by weight thermally bondable synthetic fibers, bonding can be achieved with thermal bonds. When the upper and lower layers 280, 282 are tissue layers and are hydrogen bonded using water to middle layer 284, unexpectedly good “core utilization” is realized. “Core utilization” is the percentage of the total capacity of a core that can be absorbed in a demand absorbency test. This unexpected performance improvement is believed to be the result of the good liquid distribution achieved with a high density, non-gel blocking central fibrous layer 284, and using tissue layers 280, 282 that are intimately bonded to the fibers of the inner layers 284 of the absorbent core 28. It is especially preferred in the present invention to use a dry process for forming the absorbent core 28. That is, the SAP, tissue layers, and fibrous material all are formed into the absorbent core without saturation with water or other solvent.

The absorbent article preferably contains the absorbent core 28 disposed between backsheet 24 and topsheet 26. At least one additional layer 240, (FIG. 2) also may disposed anywhere between backsheet 24 and topsheet 26. The at least one additional layer can be any layer selected from a fluid acquisition layer, a distribution layer, an additional fibrous layer optionally containing SAP, a wicking layer, a storage layer, or combinations and fragments of these layers. Such layers may be provided to assist with transferring fluids to the absorbent core 28, handling fluid surges, preventing rewet, containing absorbent material, improving core stability, or for other purposes. Skilled artisans are familiar with the various additional layers that may be included in absorbent article, and the present invention is not intended on being limited to any particular type of materials used for those layers. Rather, the invention encompasses all types of wicking layers, all types of distribution layers, etc., to the extent that type of layer 240 is utilized.

One element that is useful as an additional layer 240 in the absorbent article 10 of the invention is a fluid acquisition layer, or fluid handling layer. The fluid acquisition layer 240 typically comprises a hydrophilic fibrous material, and serves to quickly collect and temporarily hold discharged body fluid. A portion of discharged fluid may, depending upon the wearer's position, permeate the acquisition layer 240 and be absorbed by the central fibrous layer 284 in the area proximate to the discharge. However, since fluid is frequently discharged in gushes, the central fibrous layer 284 in such area may not absorb the fluid as quickly as it is discharged. Therefore, the fluid acquisition layer 240 hereof also facilitates transport of the fluid from the point of initial fluid contact to other parts of the absorbent core 28. In the context of the present invention, it should be noted that the term “fluid” includes, but is not limited to, liquids, urine, menses, perspiration, and water based body fluids.

The function of the fluid acquisition layer 240 is relatively important. The fluid acquisition layer 240 preferably has sufficient capillary suction to more fully drain the topsheet 24 and yet not exhibit excessive fluid retention to make it difficult for the underlying layer (e.g., central fibrous layer 284) to desorb the acquisition layer 240. The acquisition layer 240 may be comprised of several different materials including nonwoven or woven webs of synthetic fibers including polyester, polypropylene, or polyethylene, natural fibers including cotton or cellulose, blends of such fibers, foams, fluff pulp, apertured films, or any equivalent materials or combinations of materials.

Another useful layer 240 for use in the absorbent article 10 of the invention includes a fluid distribution layer 240. Fluid distribution layer 240 of the invention can include any combination or all of three basic components: chemically stiffened, twisted, and curled bulking fibers, high surface area fibers, and binder fibers. In a preferred embodiment of the invention, fluid distribution layer 240 comprises from about 20% to about 80% of the chemically stiffened, twisted, and cured fibers, from about 10% to about 80% of a high surface area fiber, and from 0% to about 50% of a thermoplastic binding means for increasing physical integrity of the web. All percentages herein refer to weight percentages based on total dry web weight. Preferably, the fluid distribution layer 240 will comprise between about 45% and about 60% of chemically stiffened, twisted, and cured fibers, between about 5% and about 15% of a hot melt fibrous binding means, and between about 30% and about 45% high surface area cellulose binding means. More preferably, the fluid distribution layer 240 comprises about 10% thermoplastic binding means, about 45% chemically stiffened, twisted, and cured fibers, and about 45% high surface area fibers.

Chemical additives also can be used as binding means, and are incorporated into the acquisition/distribution layer at levels typically of about 0.2% to about 2.0%, dry web weight basis. The three basic fiber components are described in greater detail in U.S. Pat. No. 5,549,589, the disclosure of which is incorporated by reference herein in its entirety, and in a manner consistent with this disclosure.

Fluid distribution layer 240 also may be comprised of non-woven or woven webs of synthetic fibers, natural fibers, foams, carded, thermal bonded materials, and the like.

Another useful layer in the absorbent article 10 of the invention includes a storage layer 240. Such storage layers 240 typically have limited transport and wicking capabilities but high storage or retention capacity, and rely upon the central fibrous layer 284 to distribute incoming fluid over a larger area. It is preferred to dispose storage layer 240 between absorbent core 28 and backsheet 26, or between upper and lower tissue layers 280, 282.

Storage layers or members 240 may be of generally conventional design and composition, selected with regard to the particular application. The storage layer or member 240 may be monolayer or multilayer, homogeneous or stratified, profiled or uniform, etc. Materials suitable for use in such storage layers 240 may be natural or synthetic in origin, woven, non-woven, fibrous, cellular, or particulate, and may include particles, layers, or regions of absorbent polymeric gelling materials. Other preferred materials include fluff pulp and SAP composites, either air laid or wet laid, and high capacity resilient foam materials. Storage layer 240 may also have any desired size and/or shape as may prove suitable for a particular application, including square, rectangular, oval, elliptical, oblong, etc. They may also take on a three-dimensional shape or may be substantially planar in nature.

Another useful layer 240 in absorbent article 10 is a wicking layer 240. Wicking layers usually have both fluid acquisition and fluid distribution properties. For example, vertical wicking, which is in general the ability to transport fluids vertically from the topsheet 24 to the absorbent core 28, is related in many respects to fluid acquisition. Horizontal wicking, which is in general the ability to transport fluids along the horizontal 100 and vertical 102 axes of FIG. 1, is related in many respects to fluid distribution. Any conventional wicking materials can be used for the wicking layer 286 of the invention. High internal phase emulsion (HIPE) foams such as those disclosed in U.S. Pat. No. 5,650,222 can be used, braided materials such as those disclosed in H1,585, and other conventional fibrous and strand materials can be used. The disclosures of U.S. Pat. No. 5,650,222 and H1,585 are incorporated by reference here in their entirety, and in a manner consistent with the present invention.

Wicking layer 240 also may be comprised of two or more sublayers containing absorbent materials with differing wicking characteristics. Any of the materials discussed in this context can be used for any and all of the wicking layers 240. In accordance with the embodiment of the invention discussed immediately above, the wicking layer 240 may include a first member that is made of a material that is capable of rapidly transferring, in the z-direction (e.g., orthogonal to the plane formed by horizontal 100 and vertical 102 axes of FIG. 1), body fluid that is delivered to topsheet 24. The first member may be designed to have a dimension narrower than the dimension of the absorbent core 28. In this regard, the sides of the first member preferably are spaced away from the longitudinal sides of the absorbent core 28 so that body fluid is restricted to the area within the periphery of the first member, before it passes down and is absorbed into central fibrous layer 284 (or the second member of the wicking layer 240). This design is believed to enable the body fluid to be combined in the central area of the absorbent core 28 and to be wicked downward so that a greater quantity of the central fibrous layer 284 can be utilized.

A suitable material for use as a first member having high wicking capacity in the z-direction, is a material available from Kimberly-Clark Corporation, in Neenah, Wis. known as PRISM. PRISM is described in U.S. Pat. No. 5,336,552, which is hereby incorporated by reference in its entirety, and in a manner consistent with this disclosure. PRISM generally is a nonwoven fabric and comprises extruded multicomponent polymeric strands including first and second polymeric components arranged in substantially distinctive zones across the cross-section of the multicomponent strands and extending continuously along the length of the multicomponent strands. Preferably, the strands are continuous filaments which may be formed by spunbonding techniques. The second component of the strands constitutes at least a portion of the peripheral surface of the multicomponent strands continuously along the length of the multicomponent strands and includes a blend of a polyolefin and an ethylene alkyl acrylate copolymer. Bonds between the multicomponent strands may be formed by the application of heat.

More specifically, the first polymeric component of the multicomponent strands is present in an amount of from about 20 to about 80 percent by weight of the strands, and the second polymeric component is present in an amount from about 80 to about 20 percent by weight of the strands. Preferably, the first polymeric component of the multicomponent strands is present in an amount of from about 40 to about 60 percent by weight of the strands and the second polymeric component is present in an amount from about 60 to about 40 percent by weight of the strands.

The term “strand” as used herein refers to an elongated extrudate formed by passing a polymer through a forming orifice such as a die. Strands include fibers, which are discontinuous strands having a definite length, and filaments, which are continuous strands of material. The nonwoven fabric of the present invention may be formed from staple multicomponent fibers. Such staple fibers may be carded and bonded to form the nonwoven fabric. Preferably, however, the nonwoven fabric of the present invention is made with continuous spunbond multicomponent filaments which are extruded, drawn and laid on a traveling forming surface.

The types of nonwoven materials that may be employed in any of the wicking layers 240 of the invention include powder-bonded-carded webs, infrared bonded carded webs, and through-air-bonded-carded webs. The infrared and through-air bonded carded webs can optionally include a mixture of different fibers, and the fiber lengths within a selected fabric web may be within the range of about 1.0 to 3.0 inch and an average bulk density of about 0.02 g/cc to about 0.06 g/cc.

The first member of wicking layer 240 also may be a nonwoven fibrous web which includes about 75 percent polyester fibers of at least 6 denier, such as PET (polyethylene terephthalate) fibers available from Celanese AG. The polyester fibers have a length ranging from about 1.5 to 2.0 inches in length. The remaining 25 percent of the fibrous web can be composed of bicomponent binder fibers of not more than 3 denier, and preferably about 1.5 denier. The bicomponent fiber length ranges from about 1.5 to 2 inches. Suitable bicomponent fibers are wettable, polyethylene/polypropylene bicomponent fiber, available from Chisso, a business having offices located in Osaka, Japan. The bicomponent fiber can be a composite, sheath-core type with the polypropylene forming the core and polyethylene forming the sheath of the composite fiber. The polyester fibers and bicomponent fibers generally are homogeneously blended together and are not in a layered configuration. The fibers can be formed into a carded web which is thermally bonded, such as by through-air bonding or infrared bonding.

The second member of wicking layer 240 may be positioned vertically below the first member, and it preferably has a higher wicking capacity along the longitudinal 100 and vertical 102 axes of FIG. 1, than the first member. Preferably, the second member has a wicking capacity at least three time greater than the first member. The second member can be equal in width to the first member, but preferably will be wider. It is preferred that the width of the wicking layer 240 in general be the same as or greater than the width of central fibrous layer 284.

The second member can be a hydrophilic material formed from various types of natural or synthetic fibers including cellulose fibers, surfactant treated meltblown fibers, wood pulp fibers, regenerated cellulose, cotton fibers or a blend of other fibers. Preferably, the second absorbent member is a material described in U.S. Pat. No. 4,100,324, and is generally known as coform. Coform is available from the Kimberly-Clark Corporation located in Neenah, Wis. and is generally a nonwoven material having a fabric-like finish and is made up of an airform matrix of thermoplastic polymeric fibers and a multiplicity of individualized wood pulp fibers. The thermoplastic fiber polymers generally have an average diameter of less than 10 microns with the individualized wood pulp fibers dispersed throughout the matrix and serving to space these microfibers from each other. The material is formed by initially utilizing the primary air stream with the meltblown microfibers and the secondary air stream containing wood pulp fibers and merging the two under turbulent conditions to form an integrated air stream along a forming surface. The fiber-like appearance of this material provides a visual appealing absorbent. Also inherent in the coform material is increased resiliency compared to conventional cellulosic absorbents.

Other suitable materials for use as wicking layer 240 include high-density air laid fluff pulps, high-density wet laid fluff pulp, and multi-groove fibers such as 4DG deep groove fiber.

Various combinations of any of the above-mentioned layers also may be used as the at least one additional layer 240. For example, additional layer 240 may be comprised of a combination of a wicking layer and a distribution layer. Hence, the additional layer 240 will have both wicking and distribution properties. Skilled artisans will be capable of designing additional layers 240 to have desired properties by combining various layer attributes, or by fragmenting the layer, using the guidelines provided herein.

The dimensions of additional layer(s) 240 may be the same as or different from the dimensions of central fibrous layer 284 and/or upper layer 280 and lower layer 282. It is preferred that additional layer(s) 240 have a width in the lateral direction (102) of anywhere from about 10 mm to about 100 mm, and preferably from about 25 mm to about 80 mm.

It is contemplated in the present invention that the absorbent core 28 be folded as it is disposed between the topsheet 24 and backsheet 26. Absorbent core 28 can be folded in any suitable manner, including any and all of those disclosed in U.S. Pat. No. 6,068,620. Those skilled in the art will appreciate that the absorbent core 28 can be folded such that the adjacent sides are touching one another, or so that channels are formed in certain areas. For example, the absorbent core 28 can be folded in the form of a “C” where the curled ends may be spaced apart to form a channel there between, and the lower edges of the curled ends may be disposed adjacent the upper edges of the bottom portion of the folded article. Alternatively, another absorbent material, or another absorbent core 28 may be disposed in the space formed by the standard “C” fold. The same considerations may be given to the “G” fold and the “U” fold where the spaces formed by these folds may be filled with another absorbent material, another absorbent core 28, or the folds may be made tight enough so that little or no space is formed. Other possible arrangements include a “Z” fold, and a pleated absorbent core 28, as will be appreciated by those skilled in the art.

The absorbent core 28 of the preferred embodiments is particularly ideal for narrow crotch diapers and training pants. Narrow crotch training pants either must typically sacrifice absorbent capacity at the narrowed portion as a result of reduced absorbent surface area, or must alternatively provide a thicker absorbent core to compensate for the reduced surface area. As the thickness of the core increases, comfort, fit and wearability decrease. By using the high absorbency absorbent core 28 of the preferred embodiments in a narrow crotch absorbent garment, the absorbent capacity through the central crotch area is not sacrificed while comfort,fit and wearability are improved. In addition, the thermal transmittance of the absorbent garment is enhanced by using the thin absorbent core 28 of the invention.

Thermal transmittance, or heat transfer, is an important factor when considering the comfort of the absorbent garment. The comfort of an article of clothing can be measured analytically, which is extremely important in measuring the comfort of an absorbent garment worn by an infant who is incapable of providing sage subjective evidence of comfort. There are a variety of methods available to analytically measure the comfort of an article of clothing, including the sweating hot plate apparatus disclosed in U.S. Pat. No. 5,749,259, the disclosure of which is incorporated by reference here in its entirety. Dry hot plates also can be used to measure heat transfer and other thermal transmittance characteristics. In the examples below, the following Test Conditions were used to measure the thermal transmittance characteristics.

Test Conditions

### Large Hot Plate Heat Transfer (watts/m² ° C.)

Heat transfer makes it possible to predict the body heat that will flow from the skin surface through the material into the surrounding atmosphere. Heat and moisture transfer properties are key properties affecting clothing comfort. These thermal properties were analyzed using a Holometric Guarded Hot Plate Thermal Measuring System. A Tenny Model T30 chamber was used to obtain and maintain the required ambient conditions. The large hot plate enabled measuring the intrinsic thermal resistance (Rcf) for a 20 in.×20 in. sample.

The testing protocol was carried out in accordance with the requirements set out in ASTM F1868-98 Standard Test Method for Thermal and Evaporative Resistance of clothing Materials using a Sweating Hot Plate; Part C. The specifics of the test and allowable refinements are as follows:

For a given replicate the temperatures, humidity, voltage, and current were measured at 1 minute intervals.

After ten measurements, the values were averaged and recorded. The test continued until six consecutive average values met the method requirement of less than a 3% drift per hour. In the examples below, the maximum-minimum difference for Rct were nominally less than 2 percent.

Rcf=the average intrinsic thermal resistance of the sample.

Rcf is determined by subtracting the average dry bare plate resistance (Rcbp) from the average of the total thermal resistance (Rct) of the specimen tested.

Rct=total thermal resistance of the specimen and surface air layer (° C. m²/watts). It can be determined in accordance with the following equation:

$$Rct ≡ \frac{( Ts - Ta ) A}{H}$$

where:

Ts is the temperature of the skin surface;

Ta is the temperature of the ambient environment;

A is the area of the sample; and

H is the heat transfer rate due to heat (watts/m² ΔT)

The test typically is conducted such that the area is constant (e.g., a 20 in.×20 in. sample), and Ta is constant. Knowing the heat transfer rate of the bare plate, the thermal resistance of the sample can be determined by subtracting the bare plate thermal resistance from the total thermal resistance.

### Thermolabo Test—Small Hot Plate Heat Transfer and clo

Clo is a unit of thermal resistance that indicates the insulating ability of the test material. The value is derived from a measure of dry heat transfer (e.g., non-sweating hot plate). Materials having higher do values provide wearers with more thermal protection, or insulation. A clo value of 1 represents a typical man's business suit expected to maintain thermal comfort for a person in a normal indoor environment. Requirements vary from 0.5 clo for summer wear to 4-5 clo for outdoor winter clothing. Humans are capable of perceiving a difference of about 0.1 clo. Given the fact that most infants remain indoors, and if taken outdoors, typically are covered with insulating clothing, the clo values for infant absorbent garments should be low. This holds true for other absorbent garments, especially those typically worn underneath other clothing.

The “thermolabo” is one of a group of laboratory instruments developed by Dr. Sueo Kawabata. Kawabata, S., et al., “Applications of the New Thermal Tester ‘Thermolabo’ to the Evaluation of Clothing Comfort,” in OBJECTIVE MEASUREMENTS: APPLICATIONS TO PRODUCT DESIGN AND PROCESS CONTROL., Eds. S. Kawabata et al., The Textile Machinery Society of Japan (1985). The thermolabo consists of three components including: (i) a box containing a thin copper heat capacitor fitted with a temperature sensing device used for measuring the amount of heat and rate of heat flow through fabric specimens during testing; (ii) a water-box with constant temperature water flow to provide a constant temperature base needed for the testing; and (iii) an insulated hot plate fitted into a box with preset temperature control possibilities.

The ability of moisture and heat to permeate through fabrics is one of the factors to consider in determining comfort. The thermal property measurements of qₘₐₓ, conductance, and dry and wet heat transfer may be measured using the Thermolabo II instrumentation. The standard specimen size for these measurements is smaller than the large hot plate, typically on the order of about 5 in.×5 in. The specimens are tested for three repetitions, each of the tests being described in more detail below.

### Heat Transfer

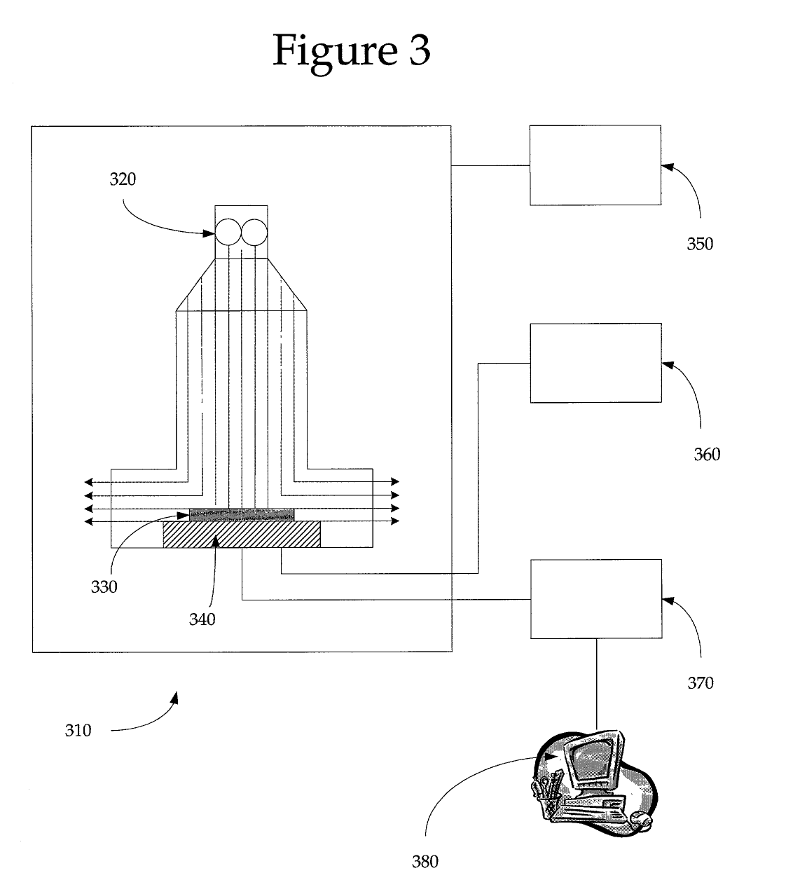

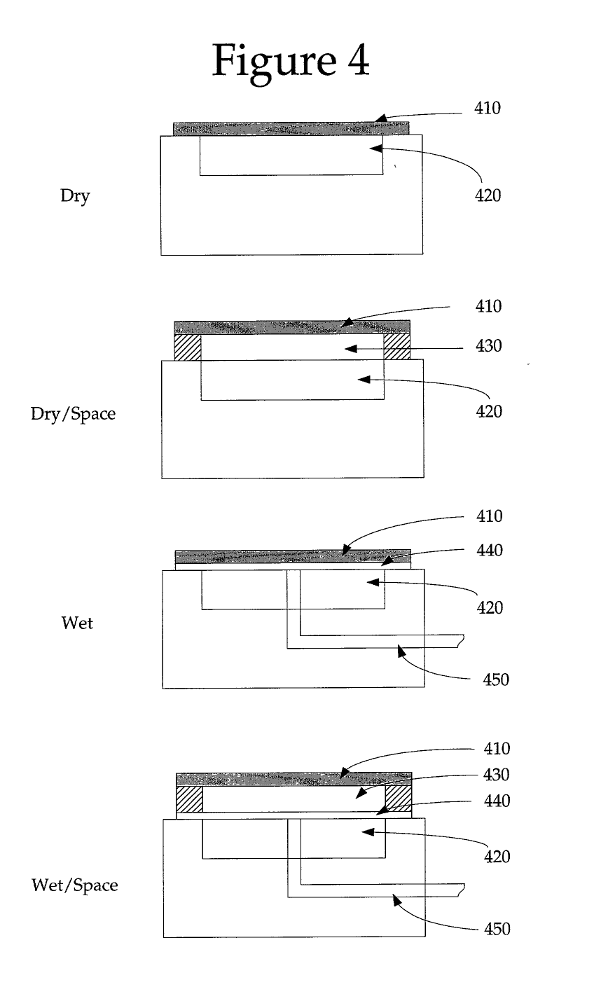

The heat an moisture properties of the 5 in.×5 in. samples are analyzed using a system consisting primarily of two parts: (i) an environmental control chamber (FIG. 3); and (ii) a component to simulate the skin/body (configurations in FIG. 4).

### Environmental Chamber

The environmental chamber used to obtain the required test conditions was a Tabai ESPEC's Platinous Lucifer Model PL-2G, with programmable low temperature and humidity. A rough schematic of such a system is illustrated in FIG. 3. The environmental chamber 310 houses a Lucite plastic sub-chamber 320 that provides precise control of air velocity. A skin simulating guarded hot plate 340 with sweating capabilities via sweating micropump 360 is positioned inside the air chamber. The sample 330 typically is placed directly on the guarded hot plate 340 for dry measurements, or can be configured in any of the configurations illustrated in FIG. 4. For the examples described below, an air current of 20 cm/sec. impinges vertically on the surface of the guarded hot plate 340, as shown by the arrows in FIG. 3.

The temperature and humidity can be controlled within chamber 310 via a computer programmable temperature and humidity controller 350. The thin copper heat capacitor fitted with a temperature sensing device can be detected in the controller amplifier detector digital indicator 370, which in turn is programmable and controllable via computer 380.

### Simulated Skin Models

Four simulated skin models are depicted in FIG. 4: (i) dry; (ii) dry/air space; (iii) wet; and (iv) wet/air space. For the dry model, a guarded hot plate 420 is used as a heat source, and the specimen 410 is placed directly on it, as shown at the top of FIG. 4. For the wet model, (third from the top in FIG. 4) simultaneous heat and moisture transfer is measured using a sweating hot plate featuring four simulated sweating glands supplying water to the heated surface at the rate of about 0.077 ml/min/gland. The water flow is controlled using a peristaltic micropump 360 (FIG. 3) to deliver water to the hot plate via line 450, while the surface of the hot plate 420 is covered with a highly wettable and dimensionally stable skin simulating membrane 440 to allow the water to spread rapidly and evenly over the surface. Heat transfer from the plate 420 through the sample test fabric 410 is determined in Watts/m² ° C.

The dry/air space model is illustrated by the second from the top drawing in FIG. 4. Here, the specimen 410 is positioned on top of the hot plate 420 with an air space 430 between the specimen and the surface of the hot plate 420. The rate of heat loss of the hot plate 420 is measured. The wet/air space model is illustrated by the bottom drawing in FIG. 4. Here, skin simulating membrane 440 is positioned on the hot plate 420 and wetted by simulated sweat glands through line 450, only this time, an air space 430 separates skin simulating membrane 440 from the specimen 410.

The following equation is used to calculate the do values:

clo(wattts/m²)=(1/Dry Heat Transfer Rate)/0.155

where Dry Heat Transfer Rate is in watts/m² ° C.

Thus, the do values reported will represent the do for both the sample tested and the bare plate. To determine the do value for the sample alone, one need only subtract the bare plate do value from the total do value. The thermal resistance also can be determined by the thermal resistance equation presented earlier.

The invention now will be explained with reference to the following examples.

### EXAMPLES

A variety of samples were tested for thermal resistance using the testing procedures outlined above. In the first example, both 20 in.×20 in. samples and 5 in.×5 in. samples were used, and in the second example, only 5 in.×5 in. samples were used.

### Example 1

A diaper was prepared in a conventional manner, with the exception that the absorbent core was prepared in the following manner to have the following characteristics. A series of absorbent cores were prepared by depositing approximately 3 grams per core of continuous filament cellulose acetate tow fiber available from Celanese Acetate, Charlotte, N.C., which had been opened, on a tissue having a basis weight of 16 grams/m² (gsm). The tissue first was treated with adhesive and the non-treated side was contacted with a rotating drum having a vacuum chamber disposed therein to draw the tissue paper onto the drum. The opened cellulose acetate tow fibers then were deposited on the adhesive-treated side of the tissue paper.

Approximately 15 grams per core of P-7700 SAP, commercially available from BASF AG, Ludwigshafen, Germany then were deposited on the opened cellulose acetate tow fibers and tissue as the components rotated on the rotating drum. A second tissue paper having a basis weight of 16 gsm, not treated with adhesive, then was applied over the SAP, cellulose acetate tow and first tissue paper to form the absorbent core unit, which then was pressed between two nip rollers. The opened ends of the upper and lower tissue layers were sealed with adhesive, and the absorbent cores cut to length. The dry formed cores had a basis weight of about 790 gsm, and a density of about 0.095 g/cm³, and were designated as core sample A.

Conventional diapers were prepared in a manner identical to those used to prepare diapers commercially available from Paragon Trade Brands, Norcross, Georgia, except that instead of the conventional absorbent cores, core sample A was used during the assembly. For the large hot plate test, portions of the diapers that included the absorbent core were cut from several diapers to make a 20×20 sample. The portions were cut from center of the training pants products so that they essentially included a non-woven topsheet material, a fluid transfer layer, (40 gsm carded thermal bonded transfer layer), the absorbent core, and a non-woven backsheet. The samples were designated Sample A.

Comparative samples were obtained in the same manner as described above, except that large training pants TP2001, available from Paragon Trade Brands, Norcross, Georgia were used. The absorbent cores had a basis weight of about 979 gsm, and a density of about 0.13 g/cm³. The samples, as cut from the training pant TP2001 had a top sheet, a transfer layer that was a carded, thermal-bonded transfer layer (40 gsm basis weight), absorbent core, and backsheet. These samples were designated Sample B.

The heat transfer characteristics then were determined using the Large Hot Plate Heat Transfer protocol outlined in the Test Methods section above. The intrinsic thermal resistance of Samples A and B are reported in table 1 below.

Additional samples were cut from the same materials used to make Samples A and B above, only these samples were cut to provide a 5 in.×5 in. sample. The respective samples were designated as samples A′ and B′. The clo values were determined on samples A′ and B′ using the Thermolabo protocol described in the Test Methods section above. The results are found in Table 2 below:

TABLE 1

| Sample | Rcf (Watts/m² ° C.) | clo (w/m²) |
|---|---|---|
| Bare plate | — | 0.4761 |
| A | 0.1788 | 1.63 |
| B | 0.2455 | 2.06 |

— indicates that no values were obtained for Rcf for the bare plate.

TABLE 2

| Sample | clo (w/m²) |
|---|---|
| Bare plate | 0.321 |
| A′ | 1.50 |
| B′ | 2.05 |

The above examples reveal that the absorbent articles made using the improved convection cores of the present invention provide more comfort than conventional absorbent articles containing conventional cores. The clo values reveal that the inventive low convection cores have a clo value of about 1.179 (clo (A)−clo (bare plate) or 1.50−0.321), whereas conventional absorbent articles have do values of about 1.73. The intrinsic thermal resistance values (rcf reported in Table 1 for the inventive samples also was significantly lower than the intrinsic thermal resistance of conventional samples (e.g., about a 27% improvement).

### Example 2

A diaper was prepared in a conventional manner as in Example 1 above, with the exception that the absorbent core was prepared in accordance with the procedures outlined above in Example 1 for core sample A. Core sample A had a basis weight of about 790 gsm, and a density of about 0.095 g/cm³, which is the same as that used in Example 1. A 5 inch by 5 inch sample was removed from the central portion of the diaper, whereby the sample included a non-woven topsheet, a fluid transfer layer, the absorbent core, and a non-woven backsheet. This sample was designated as Sample A.

Commercially available diapers of similar size and quality to Sample A were obtained, and the same size 5 in.×5 in. samples were removed from the central portion of these diapers. The densities and basis weights for the absorbent cores for these diapers are provided in Table 3 below. The samples were designated by letters H, P, and L.

The thermal resistance (clo) was determined for Samples A, H, P, and L by using the Thermolabo procedures outlined in the above Test Methods section. The results are found in Table 2.

TABLE 3

| | | Basis Weight | |
|---|---|---|---|
| Sample | Density (g/cm³) | (g/m²) | clo (W/m²) |
| Bare Plate | — | — | 0.326 |
| A | 0.095 | 790 | 1.704 |
| H | 0.12 | 1013 | 2.509 |
| L | 0.11 | 1031 | 2.532 |
| P | 0.11 | 1015 | 2.336 |

— indicates that no values were obtained.

It is clear to see from the table above that absorbent articles made with the absorbent cores of the present invention had significantly lower thermal resistance that conventional absorbent articles. Indeed, the clo value for the 5 in.×5 in. samples of the invention was about 1.374, whereas the comparative samples all had cIo values above 2.0. The absorbent articles of the present invention therefore have a greater thermal transmittance (e.g., greater heat loss), than conventional absorbent articles. Indeed, the absorbent articles of the invention provide up to a 33% decrease in thermal resistance, providing unexpectedly improved comfort.

Other embodiments, uses, and advantages of the invention will be apparent to those skilled in the art from consideration of the specification and practice of the invention disclosed herein. The specification should be considered exemplary only, and the scope of the invention is accordingly intended to be limited only by the following claims.

What is claimed is:

1. An absorbent article comprising:
a topsheet;
a backsheet,;
an absorbent core disposed between the topsheet and the backsheet;
wherein the absorbent article has a thermal resistance (clo) of less than about 1.7 watts/m², as measured in a Thermolabo apparatus.

2. The absorbent article of claim 1, whereby the topsheet and the backsheet form a first waist region, a second waist region longitudinally opposite the first waist region, and a crotch region there between, and the absorbent article further comprises
at least one fastening element attached to a lateral edge of the first waist region; and
one or more target devices attached to the article in the second waist region, where at least one fastening element and the one or more target devices are capable of attaching to one another, the one or more target devices being located so that the first waist region and second waist region of the garment may be joined to one another to secure the garment on a wearer.

3. The absorbent article of claim 1, further comprising elastic leg gathers comprising one or more elastic materials disposed adjacent a lateral edge of the crotch region, and standing leg gathers disposed on the topsheet adjacent a lateral edge of the crotch region.

4. The absorbent article of claim 2, wherein the at least one fastening element comprises a hook portion of a hook and loop fastener and the one or more target devices comprise the loop portion of a hook and loop fastener.

5. The absorbent article of claim 2, wherein the at least one fastening element is an adhesive tape and the one or more target devices comprise a tape receiving surface.

6. The absorbent article of claim 2, wherein the at least one fastening element is comprised of a pair of laterally extending tabs disposed on the lateral edges of the first waist region, whereby the laterally extending tabs each include at least one fastening element.

7. The absorbent article of claim 1, further comprising a fluid acquisition layer disposed between the topsheet and the backsheet.

8. The absorbent article of claim 1, further comprising a distribution layer disposed between the topsheet and the backsheet.

9. The absorbent article of claim 1, further comprising a wicking layer disposed between the topsheet and the backsheet.

10. The absorbent article of claim 1, further comprising a storage layer disposed between the topsheet and the backsheet.

11. The absorbent article of claim 1, further comprising a fragmented layer disposed between the topsheet and the backsheet.

12. The absorbent article of claim 1, further comprising a combination of a wicking layer and a distribution layer disposed between the topsheet and the backsheet.

13. The absorbent article of claim 1, wherein the absorbent core comprises:
an upper layer;
a lower layer; and
a central fibrous layer disposed between the upper layer and the lower layer, the central fibrous layer comprising a mixture of at least a fibrous material and superabsorbent polymer (SAP).

14. The absorbent article of claim 1, wherein the absorbent core has a density within the range of from about 0.05 to about 0.45 g/cm³.

15. The absorbent article of claim 1, wherein the absorbent core has a basis weight within the range of from about 650 to about 1350 g/cm².

16. The absorbent article of claim 13, wherein the SAP is selected from the group consisting of a water swellable polymer of water soluble acrylic or vinyl monomers crosslinked with a polyfunctional reactant, a starch modified polyacrylic acid, a hydrolyzed polyacrylonitrile, alkali metal salts of hydrolyzed polyacrylonitrile, and mixtures thereof.

17. The absorbent article of claim 16, wherein the SAP is a starch grafted polyacrylate sodium salt.

18. The absorbent article of claim 13, wherein the fibrous material is selected from the group consisting of a crimped tow of cellulose acetate or polyester, a low-density roll good, a carded web, and mixtures or combinations thereof.

19. The absorbent article of claim 18, wherein the absorbent core further comprises from about 1-5% of a thermally bondable fiber.

20. The absorbent article of claim 18, wherein the fibrous material is a crimped tow of cellulose acetate.

21. The absorbent article of claim 13, wherein the central fibrous layer comprises from about 50% to about 95% by weight super absorbent polymer (SAP), and has a SAP efficiency of at least 80%.

22. The absorbent article of claim 13, wherein the central fibrous layer further comprises particulate additives.

23. The absorbent article of claim 22, wherein the particulate additives comprise insoluble, hydrophilic polymers having particle diameters of 100 μm or less.

24. The absorbent article of claim 22, wherein the particulate additives are selected from the group consisting of potato, corn, wheat, and rice starches, and partially cooked or modified starches.

25. The absorbent article of claim 1, wherein the absorbent article has an intrinsic thermal resistance (Rcf) of less than about 0.25° C. m²/Watts, as measured on a 20×20 inch sample in a Thermolabo apparatus.

26. The absorbent article of claim 25, wherein Rcf is less than about 0.17° C. m²/Watts.

27. The absorbent article of claim 1, wherein the absorbent article has a clo of less than about 1.65 watts/m².

28. The absorbent article of claim 27, wherein the clo is less than about 1.40 watts/m².

29. The absorbent article of claim 1, wherein the absorbent core has a thickness within the range of from about to about 5 to about 20 mm.

30. A method of making an absorbent article comprising:
a) preparing a topsheet and a backsheet;
b) preparing an absorbent core by dispersing superabsorbent polymer particles (SAP) within a fibrous matrix; and
c) disposing the absorbent laminate core between the topsheet and the backsheet,
wherein the absorbent article has a thermal resistance (clo) of less than about 1.7 watts/m², as measured in a Thermolabo apparatus.

31. The method of claim 30, wherein preparing the absorbent core comprises:
supplying an upper layer;
supplying a lower layer; and
preparing a central fibrous layer by intimately mixing at least a fibrous material and superabsorbent polymer (SAP) particles; and
disposing the central fibrous layer between the upper layer and the lower layer.

32. The method of claim 30, wherein the absorbent core has a density within the range of from about 0.05 to about 0.45 g/cm³.

33. The method of claim 1, wherein the absorbent core has a basis weight within the range of from about 650 to about 1350 g/cm².

34. The method of claim 31, wherein the SAP is selected from the group consisting of a water swellable polymer of water soluble acrylic or vinyl monomers crosslinked with a polyfunctional reactant, a starch modified polyacrylic acid, a hydrolyzed polyacrylonitrile, alkali metal salts of hydrolyzed polyacrylonitrile, and mixtures thereof.

35. The method of claim 34, wherein the SAP is a starch grafted polyacrylate sodium salt.

36. The method of claim 31, wherein the fibrous material is selected from the group consisting of a crimped tow of cellulose acetate or polyester, a low-density roll good, a carded web, and mixtures or combinations thereof.

37. The method of claim 31, wherein the absorbent core further comprises from about 1-5% of a thermally bondable fiber.

38. The method of claim 36, wherein the fibrous material is a crimped tow of cellulose acetate.

39. The method of claim 31, wherein the central fibrous layer comprises from about 50% to about 95% by weight super absorbent polymer (SAP), and has a SAP efficiency of at least 80%.

40. The method of claim 31, wherein the central fibrous layer further comprises particulate additives.

41. The method of claim 40, wherein the particulate additives comprise insoluble, hydrophilic polymers having particle diameters of 100 μm or less.

42. The method of claim 41, wherein the particulate additives are selected from the group consisting of potato, corn, wheat, and rice starches, and partially cooked or modified starches.

43. The method of claim 30, wherein the absorbent article has an intrinsic thermal resistance (Rcf) of less than about 0.25° C. m²/Watts, when measured on a 20×20 inch sample in a Thermolabo apparatus.

44. The method of claim 43, wherein Rcf is less than about 0.19° C. m²/Watts.

45. The method of claim 30, wherein the absorbent article has a clo of less than about 1.65 watts/m².

46. The method of claim 45, wherein the do is less than about 1.40 watts/m².

47. The method of claim 30, wherein the absorbent core has a thickness within the range of from about 5 to 20 mm.

48. The method of claim 30, further comprising folding the absorbent core prior to disposing the core between the topsheet and the backsheet.

49. The method of claim 48, wherein the absorbent core is folded into a “C” configuration.
